(19) United States
(12) Patent Application Publication
(10) Pub. No.: US 2016/0369058 A1
(43) Pub. Date: Dec. 22, 2016

(54) DUAL MODALITIES CURING SILICONE COMPOSITIONS

(71) Applicant: Momentive Performance Materials Inc., Waterford, NY (US)

(72) Inventors: Huiping ZHANG, Clifton Park, NY (US); Stephan BOSSHAMMER, Cologn (DE); Dieter WROBEL, Leverkusen (DE); Richard ECKBERG, Ganesvoort, NY (US); Roy GRISWOLD, Ballston Spa, NY (US); Beate GANTER, Nordrhein Westfalen (DE)

(21) Appl. No.: 14/901,321

(22) PCT Filed: Jul. 10, 2014
(86) PCT No.: PCT/US2014/046090

§ 371 Date: Dec. 28, 2015

Related U.S. Application Data
(60) Provisional application No. 61/845,103, filed on Jul. 11, 2013.

(51) Int. Cl. C08G 77/12; A61Q 15/00; A61Q 19/00; A61Q 19/10; A61Q 5/12; A61Q 5/02; A61Q 5/06; A61Q 5/08; A61Q 3/02; A61Q 3/04; A61Q 19/08; A61Q 1/10; A61Q 1/12; A61Q 1/08; A61Q 1/06; A61K 8/89; A61K 47/34; C09D 183/04; A61Q 17/04

(52) U.S. Cl. CPC C08G 77/12; A61Q 17/04; A61Q 15/00; A61Q 19/007; A61Q 19/10; A61Q 5/12; A61Q 5/02; A61Q 5/06; A61Q 5/08; A61Q 3/02; A61Q 3/04; A61Q 19/08; A61Q 1/10; A61Q 1/12; A61Q 1/08; A61Q 1/06; A61K 8/89; A61K 47/34; C09D 183/04

(57) ABSTRACT

Silicone compositions are provided which comprises the reaction product of at least one hydride-functional silicone, at least one imsaturated-functional silicone and at least one epoxy or oxetane functional silicone. The silicone compositions are capable of being cured by two different curing modalities or capable of simultaneous curing utilizing those different curing modalities. The resulted silicone compositions have dramatically improved hydrophilicity, physical properties and optical properties.

### BACKGROUND OF THE INVENTION

1. Field of the Invention

This invention relates to silicone compositions, more particularly, silicone compositions capable of being cured by two different curing modalities or capable of simultaneous curing utilizing those different curing modalities. The silicone compositions possess enhanced hydrophilicity, physical properties and optical properties which can be used in applications such as transdermal patches for healthcare and pharmaceutical applications, drug delivery devices, coating, cosmetic structuring material, gasketing materials, agricultural spray, homecare products, rubbers and other applications where hydrophilicity is required, such as implants, as biomaterials or additives of biomaterials, as a support for cell cultures for tissue engineering or as vectorization agents for active ingredients; surfactant such as surface agent, emulsifier, co-emulsifier, dispersant, co-dispersant, adhesive or component of an adhesive, foam control, antifoaming agent, antidrift and anti-drift agent.

2. Description of Related Art

Silicone compositions are highly hydrophobic and present difficulties for use in applications where hydrophilicity is required, such as drug delivery devices. In some applications, for example, transdermal patches, additional hydrophilic coatings are generally needed in order to achieve a hydrophilic and lubricious silicone surface. Increasing the hydrophilicity of silicone compositions has been attempted to be increased by using various hydrophilic functional groups. However, the mechanical performance of such resultant silicones has generally not proven sufficient for applications such as transdermal patches for healthcare and pharmaceutical applications, drug delivery devices, coating, cosmetic structuring material, gasketing materials and other applications where hydrophilicity is required. Hydrophilicity can also be introduced into a silicone composition by various means such as immersion or blending with other siloxane(s). However, such processes still present various difficulties.

### SUMMARY OF THE INVENTION

In one non-limiting embodiment herein there is provided a silicone composition comprising the reaction product of:
a) at least one hydride-functional silicone having the formula (i)

MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and

M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)

M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)

wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
   where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1.

### DETAILED DESCRIPTION OF PREFERRED EMBODIMENTS

In the specification and claims herein, the following terms and expressions are to be understood as indicated herein below.

As used in the specification and including the appended claims, the singular forms “a,” “an,” and “the” include the plural, and reference to a particular numerical value includes at least that particular value unless the context clearly indicates otherwise.

Other than in the working examples or where otherwise indicated, numerical values and ranges of numerical values herein whether or not modified by such terms as “about” and “approximately” are to be understood to include the indicated value(s) and value(s) approximate thereto. When such a range is expressed, another embodiment includes from the one particular value and/or to the other particular value. Similarly, when values are expressed as approximations, e.g., by use of the modifier “about,” it will be understood that the particular value forms another embodiment.

It will also be understood that any numerical range recited herein is intended to include all sub-ranges within that range and any combination of end points of said ranges or sub-ranges.

All methods described herein may be performed in any suitable order unless otherwise indicated or clearly contrary to context. The use herein of any and all examples or exemplification language (e.g., “such as”), is intended merely to better illustrate the invention and does not pose a limitation on the scope of the invention unless otherwise claimed. No language in the specification should be construed as indicating any non-claimed element as essential to the practice of the invention.

As used herein, the terms “comprising,” “including,” “containing,” “characterized by” and grammatical equivalents thereof are inclusive or open-ended terms that do not exclude additional, unrecited elements or method steps and are also to be understood as including the more restrictive terms “consisting of” and “consisting essentially of.”

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

As used herein, the term “network” means a three dimensionally extending structure comprising interpenetrating siloxane polymer chains.

As used herein, the term “polymer” includes homopolymer, copolymer, terpolymer and higher order polymers.

As used herein, the term “monovalent” in reference to a hydrocarbon radical means that the radical is capable of forming one covalent bond per radical.

As used herein, the term “hydrocarbon radical” includes linear hydrocarbon radicals, branched hydrocarbon radicals, acyclic hydrocarbon radicals, alicyclic hydrocarbon radicals and aromatic hydrocarbon radicals.

As used herein, the term “acyclic hydrocarbon radical” means any straight chain or branched hydrocarbon radical, preferably containing from 1 to 60 carbon atoms per radical, which may be saturated or unsaturated and which may be optionally substituted or interrupted with one or more atoms or functional groups, such as, for example, carboxyl, cyano, hydroxy, halo and oxy. As long as these functional groups do not interfere with the cationic cure mechanism of the epoxide or oxirane moiety, suitable monovalent acyclic hydrocarbon radicals may include, for example, alkyl, alkenyl, alkynyl, hydroxyalkyl, cyanoalkyl, carboxyalkyl, alkyloxy, oxaalkyl, alkylcarbonyloxaalkylene, carboxamide and haloalkyl, such as, for example, methyl, ethyl, sec-butyl, tert-butyl, octyl, decyl, dodecyl, cetyl, stearyl, ethenyl, propenyl, butynyl, hydroxypropyl, cyanoethyl, butoxy, 2,5,8-trioxadecanyl, carboxymethyl, chloromethyl and 3,3,3-fluoropropyl. Suitable divalent acyclic hydrocarbon radicals include, for example, linear or branched alkylene radicals, such as, for example, methylene, dimethylene, trimethylene, decamethylene, ethylethylene, 2-methyltrimethylene, 2,2-dimethyltrimethylene and linear or branched oxalkylene radicals such as, for example, methyleneoxypropylene. Suitable trivalent acyclic hydrocarbon radicals include, for example, alkanetriyl radicals, such as, for example, 1,1,2-ethanetriyl, 1,2,4-butanetriyl, 1,2,8-octanetriyl, 1,2,4-cyclohexanetriyl and oxaalkanetriyl radicals such as, for example, 1,2,6-triyl-4-oxahexane.

As used herein the term “alkyl” means any saturated straight or branched monovalent hydrocarbon radical. In a preferred embodiment, monovalent alkyl groups are selected from linear or branched alkyl groups containing from 1 to 60 carbons per group, such as, for example, methyl, ethyl, propyl, iso-propyl, n-butyl, iso-butyl, sec-butyl, tert-butyl, pentyl, hexyl, heptyl, decyl, dodecyl.

As used herein the term “alkenyl” means any straight or branched monovalent terminally unsaturated hydrocarbon radical, preferably containing from 2 to 10 carbon atoms per radical, such as, for example, ethenyl, 2-propenyl, 3-butenyl, 5-hexenyl, 7-octenyl and ethenylphenyl.

As used herein, the term “alicyclic hydrocarbon radical” means a radical containing one or more saturated hydrocarbon rings, preferably containing from 4 to 12 carbon atoms per ring, per radical which may optionally be substituted on one or more of the rings with one or more alkyl radicals, each preferably containing from 2 to 6 carbon atoms per alkyl radical, halo radicals or other functional groups and which, in the case of a monovalent alicyclic hydrocarbon radical containing two or more rings, may be fused rings. Suitable monovalent alicyclic hydrocarbon radicals include, for example, cyclohexyl and cyclooctyl. Suitable divalent hydrocarbon radicals include, saturated or unsaturated divalent monocyclic hydrocarbon radicals, such as, for example, 1,4-cyclohexylene. Suitable trivalent alicyclic hydrocarbon radicals include, for example, cycloalkanetriyl radicals such as, for example, 1-dimethylene-2,4-cyclohexylene, 1-methylethylene-3-methyl-3,4-cyclohexylene.

As used herein, the term “aromatic hydrocarbon radical” means a hydrocarbon radical containing one or more aromatic rings per radical, which may, optionally, be substituted on the aromatic rings with one or more alkyl radicals, each preferably containing from 2 to 6 carbon atoms per alkyl radical, halo radicals or other functional groups and which, in the case of a monovalent aromatic hydrocarbon radical containing two or more rings, may be fused rings. Suitable monovalent aromatic hydrocarbon radicals include, for example, phenyl, tolyl, 2,4,6-trimethylphenyl, 1,2-isopropylmethylphenyl, 1-pentalenyl, naphthyl, anthryl, eugenol and allylphenol as well as aralkyl radicals such as, for example, 2-phenylethyl. Suitable divalent aromatic hydrocarbon radicals include, for example, divalent monocyclic arenes such as, for example, 1,2-phenylene, 1,4-phenylene, 4-methyl-1,2-phenylene, phenylmethylene. Suitable trivalent aromatic hydrocarbon radicals include, for example, trivalent monocyclic arenes such as, for example, 1-trimethylene-3,5-phenylene.

It is noted that the present inventors herein have unexpectedly discovered, in one specific embodiment, silicone compositions capable of being cured by two different curing modalities, or capable of simultaneous curing utilizing those modalities, that comprises the reaction product of at least three functionally different silicones each having at least one or two of the reactive moieties of hydride, unsaturated-bond and epoxy (or oxetane), respectively. The silicone compositions described herein possess enhanced hydrophilicity, physical properties and optical properties as compared to previously known silicones other than the reaction product described herein.

In one embodiment herein there is provided a silicone composition comprising the reaction product of at least one of the above-identified hydride-functional silicone (i), at least one of the above-identified unsaturated-functional silicone (ii) and at least one of the above-identified epoxy or oxetane functional silicone (iii) which silicones (i), (ii) and (iii) are reacted optionally in the presence of a catalyst, e.g., a platinum catalyst, to produce the silicone composition.

It will be understood herein that any known or commercially used hydride-functional silicone having at least two hydride moieties per molecule can be employed as silicone (i) herein. As indicated in formula (i), the hydride-functional silicone can have any amount of unsaturated-functional moieties, epoxy (or oxetane) functional moieties, and polyether moieties. The hydride-functional silicone can also have any amount of other functional moieties other than those specified above.

In formula (i), in one embodiment, R¹, R², R³, R⁴ R⁵ and R⁶ are each independently monovalent hydrocarbon radicals having from 1 to about 60 carbon atoms, more specifically of from 1 to about 30 carbon atoms and most specifically of from 1 to about 20 carbon atoms; R⁷, R⁸ and R⁹ are each independently hydrogen or monovalent hydrocarbon radicals having from 1 to about 60 carbon atoms, more specifically of from 1 to about 30 carbon atoms and most specifically of from 1 to about 20 carbon atoms; R¹⁰, R¹¹ and R¹² are each independently monovalent hydrocarbon radicals having from 1 to about 60 carbon atoms, more specifically of from 1 to about 30 carbon atoms and most specifically of from 1 to about 20 carbon atoms, or R^{vi}; each R^{vi} is independently a monovalent unsaturated hydrocarbon radical having from 2 to about 10 carbon atoms, more specifically of from 2 to about 8 carbon atoms and most specifically of from 2 to about 5 carbon atoms; R¹³, R¹⁴ and R¹⁵ are each independently monovalent hydrocarbon radicals having from 1 to about 60 carbon atoms, more specifically of from 1 to about 30 carbon atoms and most specifically of from 1 to about 20 carbon atoms, or R^{E}; each R^{E} is independently an epoxy or an oxetane radical having from 2 to about 60 carbon atoms, more specifically of from 1 to about 30 carbon atoms and most specifically of from 1 to about 20 carbon atoms, or one or more heteroatoms; R¹⁶, R¹⁷ and R¹⁸ are each independently a monovalent hydrocarbon radicals having from 1 to about 60 carbon atoms, more specifically of from 1 to about 30 carbon atoms and most specifically of from 1 to about 20 carbon atoms, or R^{P}; each R^{P} is independently a monovalent polyether moiety (C_{S}H_{2S}O)_{X} or a copolymer of monovalent polyether moieties (C_{S}H_{2S}O)_{X} and (C_{T}H_{2T}O)_{Y} or a higher order polymer wherein the subscripts S and T are either from 2 to about 20, more specifically of from 2 to about 15 carbon atoms and most specifically of from 2 to about 10 carbon atoms, and the subscripts X and Y are either from 1 to about 30, more specifically of from about 5 to about 30 carbon atoms and most specifically of from about 5 to about 20 carbon atoms.

In formula (i), in one embodiment, the subscripts a, b, c, d, e, f, g, h, i, j, k, m, n, p, q and r are each either zero or positive integers from 1 to about 1000, subject to the limitations that a+b+c+d+e≧2, and b+g+m≧2.

In one embodiment, the hydride-functional silicone (i) has a hydride content in the range of from about 0.3 micromoles per gram (mmol/g) to about 15 mmol/g, more specifically of from 2 mmol/g to about 15 mmol/g and most specifically of from 4 mmol/g to about 15 mmol/g.

The hydride-functional silicone (i) can have in one embodiment the formula MₐM^{H}_{b}D_{f}D^{H}_{g}TₖT^{H}ₘQᵣ wherein M, M^{H}, D, D^{H}, T, T^{H} and Q are as defined above; R¹, R², R³, R⁴, R³, R⁶, R⁷, R⁸ and R⁹ are each independently selected from the group consisting of methyl, ethyl, isopropyl, sec-butyl and tert-butyl; subscripts a, b, f, g, k, m and r are each positive integers in the range of from about 1 to about 100, more specifically of from about 5 to about 50 and most specifically of from about 10 to about 30.

It will be understood herein that any known or commercially used unsaturated-functional silicone having at least two unsaturated hydrocarbon moieties per molecule can be employed as silicone (ii) herein. As indicated in formula (ii), the unsaturated-functional silicone can have any amount of hydride-functional moieties, epoxy (or oxetane) functional moieties, and polyether moieties. The unsaturated-functional silicone can also have any amount of other functional moieties other than those specified above.

In formula (ii), in one embodiment, R¹, R², R³, R⁴ R⁵ and R⁶ are each independently monovalent hydrocarbon radicals having from 1 to about 60 carbon atoms, more specifically of from 1 to about 30 carbon atoms and most specifically of from 1 to about 20 carbon atoms; R⁷, R⁸ and R⁹ are each independently hydrogen or monovalent hydrocarbon radicals having from 1 to about 60 carbon atoms, more specifically of from 1 to about 30 carbon atoms and most specifically of from 1 to about 20 carbon atoms; R¹⁰, R¹¹ and R¹² are each independently monovalent hydrocarbon radicals having from 1 to about 60 carbon atoms, more specifically of from 1 to about 30 carbon atoms and most specifically of from 1 to about 20 carbon atoms, or R^{vi}; each R^{vi} is independently a monovalent unsaturated hydrocarbon radical having from 2 to about 10 carbon atoms, more specifically of from 2 to about 8 carbon atoms and most specifically of from 2 to about 5 carbon atoms; R¹³, R¹⁴ and R¹⁵ are each independently monovalent hydrocarbon radicals having from 1 to about 60 carbon atoms, more specifically of from 1 to about 30 carbon atoms and most specifically of from 1 to about 20 carbon atoms, or R^{E}; each R^{E} is independently an epoxy or an oxetane radical having from 2 to about 60 carbon atoms, more specifically of from 1 to about 30 carbon atoms and most specifically of from 1 to about 20 carbon atoms, or one or more heteroatoms; R¹⁶, R¹⁷ and R¹⁸ are each independently a monovalent hydrocarbon radicals having from 1 to about 60 carbon atoms, more specifically of from 1 to about 30 carbon atoms and most specifically of from 1 to about 20 carbon atoms, or R^{P}; each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, more specifically of from 2 to about 15 carbon atoms and most specifically of from 2 to about 10 carbon atoms, and the subscripts X and Y are either from 1 to about 30, more specifically of from about 5 to about 30 carbon atoms and most specifically of from about 5 to about 20 carbon atoms.

In formula (ii), in one embodiment, the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2.

In one embodiment, the unsaturated-functional silicone has a unsaturated moiety content in the range of from about 0.01 mmol/g to about 2 mmol/g, more specifically of from 0.01 mmol/g to about 1 mmol/g and most specifically of from 0.02 mmol/g to about 0.4 mmol/g.

The unsaturated-functional silicone (ii) can have in one embodiment the formula M_{a′}M^{vi}_{c′}D_{f′}Q_{r′} wherein M, M^{vi}, D and Q are as defined above; R¹, R², R³, R⁴ and R⁵ are each independently selected from the group consisting of methyl, ethyl, isopropyl, sec-butyl and tert-butyl; R¹⁰ and R¹¹ are each independently selected from the group consisting of methyl, ethyl, isopropyl, sec-butyl and tert-butyl or R^{vi}; each R^{vi} is independently selected from the group consisting of ethenyl, 2-propenyl, 3-butenyl, 5-hexenyl, 7-octenyl and ethenylphenyl; and subscripts a′, c′ f′ and r′ are each positive integer in the range of from about 1 to about 2000, more specifically of from about 50 to about 1500 and most specifically of from about 100 to about 1000.

It will be understood herein that any known or commercially used epoxy or oxetane functional siloxane can be employed as silicone (iii) herein. As indicated in formula (iii), the epoxy or oxetane functional silicone can have any amount of hydride-functional moieties, unsaturated-functional moieties, and polyether moieties. The epoxy or oxetane functional silicone can also have any amount of other functional moieties other than those specified above.

In formula (iii), in one embodiment, R¹, R², R³, R⁴ R⁵ and R⁶ are each independently monovalent hydrocarbon radicals having from 1 to about 60 carbon atoms, more specifically of from 1 to about 30 carbon atoms and most specifically of from 1 to about 20 carbon atoms; R⁷, R⁸ and R⁹ are each independently hydrogen or monovalent hydrocarbon radicals having from 1 to about 60 carbon atoms, more specifically of from 1 to about 30 carbon atoms and most specifically of from 1 to about 20 carbon atoms; R¹⁰, R¹¹ and R¹² are each independently monovalent hydrocarbon radicals having from 1 to about 60 carbon atoms, more specifically of from 1 to about 30 carbon atoms and most specifically of from 1 to about 20 carbon atoms, or R^{vi}; each R^{vi} is independently a monovalent unsaturated hydrocarbon radical having from 2 to about 10 carbon atoms, more specifically of from 2 to about 8 carbon atoms and most specifically of from 2 to about 5 carbon atoms; R¹³, R¹⁴ and R¹⁵ are each independently monovalent hydrocarbon radicals having from 1 to about 60 carbon atoms, more specifically of from 1 to about 30 carbon atoms and most specifically of from 1 to about 20 carbon atoms, or R^{E}; each R^{E} is independently an epoxy or an oxetane radical having from 2 to about 60 carbon atoms, more specifically of from 1 to about 30 carbon atoms and most specifically of from 1 to about 20 carbon atoms, or one or more heteroatoms; R¹⁶, R¹⁷ and R¹⁸ are each independently a monovalent hydrocarbon radicals having from 1 to about 60 carbon atoms, more specifically of from 1 to about 30 carbon atoms and most specifically of from 1 to about 20 carbon atoms, or R^{P}; each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, more specifically of from 2 to about 15 carbon atoms and most specifically of from 2 to about 10 carbon atoms, and the subscripts X and Y are either from 1 to about 30, more specifically of from about 5 to about 30 carbon atoms and most specifically of from about 5 to about 20 carbon atoms.

In formula (iii), in one embodiment, the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1.

In one embodiment, the epoxy or oxetane functional silicone has an epoxy or an oxetane content in the range of from about 0.1 mmol/g to about 1.5 mmol/g, more specifically of from 0.1 mmol/g to about 1 mmol/g and most specifically of from 0.2 mmol/g to about 0.7 mmol/g.

The epoxy or oxetane functional silicone (iii) can have in one embodiment the formula M_{a″}M^{E}_{d″}D_{f″}D^{E}_{i″}T_{k″}Q_{r″} wherein M, M^{E}, D, D^{E}, T and Q are as defined above; R¹, R², R³, R⁴, R⁵ and R⁶ are each independently selected from the group consisting of methyl, ethyl, isopropyl, sec-butyl and tert-butyl; R¹³, R¹⁴ and R¹⁵ are each independently selected from the group consisting of methyl, ethyl, isopropyl, sec-butyl and tert-butyl or R^{E}; each R^{E} is independently selected from the group consisting of 2-(3,4-epoxycyclohexyl)ethyl, 2-(3,4-epoxycyclopentyl)ethyl, 2-(3,4-epoxycyclohexyl)propyl and 2-(3,4-epoxycyclopentyl)propyl; subscripts a″, d″, f″, i″, k″ and r″ are each positive integer in the range of from 1 to about 2000, more specifically of from about 5 to about 1000, and most specifically of from about 10 to about 600.

It will be understood herein that any known or commercially used catalyst can be employed herein, most specifically a photo-activated platinum catalyst. In one embodiment, the photo-activated platinum catalyst is selected from the group consisting of η⁵-cyclopentadienyl platinum(IV) complexes, bis(β-diketonate) platinum(II) complexes, bis(phosphine) platinum(II) complexes, cyclooctadiene platinum(II) complexes, and mixtures thereof, more specifically trimethyl(methylcyclopentadienyl) platinum(IV) (CpPt) or platinum(I) acetylacetonate (Pt(acac)2).

In one embodiment, the photo-activated catalysts can also be activated by heat. It will be understood herein that any known or commercially used heat-activated platinum catalyst can be employed herein. The heat-activated platinum catalyst is selected from the group consisting of platinum compounds such as chloroplatinic acid, or platinum complexes such as platinum/vinylsiloxane complexes, or mixtures thereof.

It will be understood herein that any known or commercially used photo-initiator can be employed in the composition herein. In one embodiment, the photo-initiator is selected from the group consisting of iron (II) sandwich complexes including benzenecyclopentadienyliron (II) hexafluorophosphate and cyclopentadienyl iron (II) dicarbonyl dimer, or bis(4-alkylphenyl)iodonium salts of PF₆⁻ or SbF₆⁻, or mixtures thereof. The photo-initiators can trigger the epoxide ring opening reaction upon exposure to UV irradiation.

The compositions herein may further comprise additional components such as a filler, photo-sensitizer, stabilizer, inhibitor or adhesion promoter, plasticizers, flame retardants, smoke suppressants, antioxidants, lubricants, pigments, initiators, lubricants and flow control agents, antistatic agents, blowing/foaming agents, antifouling agent and any other additives known in the art to improve the physical properties of the resulting composition and any combination of these and other additional components.

It will be understood herein that any known or commercially used filler can be employed herein. The filler is selected from the group consisting of silica aerogel, fumed silica, flux-calcined diatomaceous silica, calcined diatomaceous silica, calcined kaolin, precipitated calcium carbonate, ground silica, acetylene black, alumina, carbon black, carbon molecular sieves, silver dioxide, titanium dioxide, zirconium oxide, iron oxide. In one embodiment, the silica filler is selected from the group consisting of fumed silica, precipitated silica, and mixtures thereof. In one embodiment, the silica filler can be surface-treated or left untreated. Some non-limiting examples of treatment used to treat silica filler are organoalkoxysilanes, and further such treated silica filler can be further treated with passivating agents such as those selected from the group consisting of hexamethyldisilazane, divinyltetramethydisilazane, octamethylcyclotetrasiloxane, and mixtures thereof.

It will be understood herein that any known or commercially used photo-sensitizer can be employed herein. In one embodiment, the photo-sensitizer is selected from the group consisting of polycyclic aromatic compounds such as anthracene and its derivatives, or ketone chromophores such as thioxanthone and its derivatives, or mixtures thereof.

It will be understood herein that any known or commercially used inhibitor can be employed herein. In one embodiment, the inhibitor is selected from the group consisting of β-alkynoles, or maleates, or fumarates, or vinyl-containing organosiloxanes, or phosphites, or cyanurates, or mixtures thereof.

Adhesion promoter can be used to covalently link to the silicone composition. It will be understood herein that any known or commercially used adhesion promoter can be employed herein. In one embodiment, the adhesion promoters are selected from the group consisting of multi-alkoxysilanes, which contain silicon-hydride, or vinyl, or epoxy (or oxetane) functional group.

### Applications for Embodiments of the Invention

### A. Agricultural Uses

Pesticide—Agriculture, Horticulture, Turf, Ornamental and Forestry:

Many pesticide applications require the addition of an adjuvant to the spray mixture to provide wetting and spreading on foliar surfaces. Often that adjuvant is a surfactant, which can perform a variety of functions, such as increasing spray droplet retention on difficult to wet leaf surfaces, enhance spreading to improve spray coverage, or to provide penetration of the herbicide into the plant cuticle. These adjuvants are provided either as a tank-side additive or used as a component in pesticide formulations.

Typical uses for pesticides include agricultural, horticultural, turf, ornamental, home and garden, veterinary and forestry applications.

The pesticidal compositions of the present invention also include at least one pesticide, where the composition of the present invention is present at an amount sufficient to deliver between 0.005% and 2% to the final use concentration, either as a concentrate or diluted in a tank mix. Optionally the pesticidal composition may include excipients, cosurfactants, solvents, foam control agents, deposition aids, drift retardants, biologicals, micronutrients, fertilizers and the like. The term pesticide means any compound used to destroy pests, e.g., rodenticides, insecticides, miticides, fungicides, and herbicides. Illustrative examples of pesticides that can be employed include, but are not limited to, growth regulators, photosynthesis inhibitors, pigment inhibitors, mitotic disrupters, lipid biosynthesis inhibitors, cell wall inhibitors, and cell membrane disrupters. The amount of pesticide employed in compositions of the invention varies with the type of pesticide employed. More specific examples of pesticide compounds that can be used with the compositions of the invention are, but not limited to, herbicides and growth regulators, such as: phenoxy acetic acids, phenoxy propionic acids, phenoxy butyric acids, benzoic acids, triazines and s-triazines, substituted ureas, uracils, bentazon, desmedipham, methazole, phenmedipham, pyridate, amitrole, clomazone, fluridone, norflurazone, dinitroanilines, isopropalin, oryzalin, pendimethalin, prodiamine, trifluralin, glyphosate, sulfonylurcas, imidazolinones, clethodim, diclofop-methyl, fenoxaprop-ethyl, fluazifop-p-butyl, haloxyfop-methyl, quizalofop, sethoxydim, dichlobenil, isoxaben, and bipyridylium compounds.

Fungicide compositions that can be used with the present invention include, but are not limited to, aldimorph, tridemorph, dodemorph, dimethomorph; flusilazol, azaconazole, cyproconazole, epoxiconazole, furconazole, propiconazole, tebuconazole and the like; imazalil, thiophanate, benomyl carbendazim, chlorothialonil, dicloran, trifloxystrobin, fluoxystrobin, dimoxystrobin, azoxystrobin, furcaranil, prochloraz, flusulfamide, famoxadone, captan, maneb, mancozeb, dodicin, dodine, and metalaxyl.

Insecticide, larvacide, miticide and ovacide compounds that can be used with the composition of the present invention, but not limited to, Bacillus thuringiensis, spinosad, abamectin, doramectin, lepimectin, pyrethrins, carbaryl, primicarb, aldicarb, methomyl, amitraz, boric acid, chlordimeform, novaluron, bistrifluron, triflumuron, diflubenzuron, imidacloprid, diazinon, acephate, endosulfan, kelevan, dimethoate, azinphos-ethyl, azinphos-methyl, izoxathion, chlorpyrifos, clofentezine, lambda-cyhalothrin, permethrin, bifenthrin, cypermethrin and the like.

Fertilizers and Micronutrients:

Fertilizers and micronutrients include, but not limited to, zinc sulfate, ferrous sulfate, ammonium sulfate, urea, urea ammonium nitrogen, ammonium thiosulfate, potassium sulfate, monoammonium phosphate, urea phosphate, calcium nitrate, boric acid, potassium and sodium salts of boric acid, phosphoric acid, magnesium hydroxide, manganese carbonate, calcium polysulfide, copper sulfate, manganese sulfate, iron sulfate, calcium sulfate, sodium molybdate, calcium chloride,

The pesticide or fertilizer may be a liquid or a solid. If a solid, it is preferable that it is soluble in a solvent, or the organomodified disiloxanes of the present invention, prior to application, and the silicone may act as a solvent, or surfactant for such solubility or additional surfactants may perform this function

Agricultural Excipients:

Buffers, preservatives and other standard excipients known in the art also may be included in the composition.

Solvents may also be included in compositions of the present invention. These solvents are in a liquid state at room temperature. Examples include water, alcohols, aromatic solvents, oils (i.e. mineral oil, vegetable oil, silicone oil, and so forth), lower alkyl esters of vegetable oils, fatty acids, ketones, glycols, polyethylene glycols, diols, paraffinics, and so forth. Particular solvents would be 2,2,4-trimethyl, 1-3-pentane diol and alkoxylated (especially ethoxylated) versions thereof as illustrated in U.S. Pat. No. 5,674,832 herein incorporated by reference, or n-methyl-pyrrilidone.

Cosurfactants:

Cosurfactants useful herein include nonionic, cationic, anionic, amphoteric, zwitterionic, polymeric surfactants, or any mixture thereof. Surfactants are typically hydrocarbon based, silicone based or fluorocarbon based.

Moreover, other cosurfactants, that have short chain hydrophobes that do not interfere with superspreading as described in U.S. Pat. No. 5,558,806 herein incorporated by reference are also useful. Additionally, the compositions described above are also useful as the alkyl chloride, alkyl iodide and alkyl bromide analogues, as well as the acid pairs with HCl, acetic acid, propionic acid, glycolic acid, gibberellic acid and the like. One skilled in the art understands the benefits of quaternizernization, which increases solubility and as well as makes possible potential interactions with nonionic and anionic cosurfactants.

Useful surfactants include alkoxylates, especially ethoxylates, containing block copolymers including copolymers of ethylene oxide, propylene oxide, butylene oxide, and mixtures thereof; alkylarylalkoxylates, especially ethoxylates or propoxylates and their derivatives including alkyl phenol ethoxylate; arylarylalkoxylates, especially ethoxylates or propoxylates, and their derivatives; amine alkoxylates, especially amine ethoxylates; fatty acid alkoxylates; fatty alcohol alkoxylates; alkyl sulfonates; alkyl benzene and alkyl naphthalene sulfonates; sulfated fatty alcohols, amines or acid amides; acid esters of sodium isethionate; esters of sodium sulfosuccinate; sulfated or sulfonated fatty acid esters; petroleum sulfonates; N-acyl sarcosinates; alkyl polyglycosides; alkyl ethoxylated amines: and so forth.

Specific examples include alkyl acetylenic diols (SURFONYL—Air Products), pyrrilodone based surfactants (e.g., SURFADONE—LP 100—ISP), 2-ethyl hexyl sulfate, isodecyl alcohol ethoxylates (e.g., RHODASURF DA 530—Rhodia), ethylene diamine alkoxylates (TETRONICS—BASF), ethylene oxide/propylene oxide copolymers (PLURONICS—BASF), Gemini type surfactants (Rhodia) and diphenyl ether Gemini type surfactants (e.g. DOWFAX—Dow Chemical).

Preferred surfactants include ethylene oxide/propylene oxide copolymers (EO/PO); amine ethoxylates; alkyl polyglycosides; oxo-tridecyl alcohol ethoxylates, and so forth.

In a preferred embodiment, the agrochemical composition of the present invention further comprises one or more agrochemical ingredients. Suitable agrochemical ingredients include, but not limited to, herbicides, insecticides, growth regulators, fungicides, miticides, acaricides, fertilizers, biologicals, plant nutritionals, micronutrients, biocides, paraffinic mineral oil, methylated seed oils (i.e. methylsoyate or methylcanolate), vegetable oils (such as soybean oil and canola oil), water conditioning agents such as Choice® (Loveland Industries, Greeley, Colo.) and Quest (Helena Chemical, Collierville, Tenn.), modified clays such as Surround® (Englehard Corp.), foam control agents, surfactants, wetting agents, dispersants, emulsifiers, deposition aids, antidrift components, and water.

Suitable agrochemical compositions are made by combining, in a manner known in the art, such as, by mixing one or more of the above components with the organomodified disiloxane of the present invention, either as a tank-mix, or as an “In-can” formulation. The term “tank-mix” means the addition of at least one agrochemical to a spray medium, such as water or oil, at the point of use. The term “In-can” refers to a formulation or concentrate containing at least one agrochemical component. The “In-can” formulation may then diluted to use concentration at the point of use, typically in a Tank-mix, or it may be used undiluted.

### B. Coatings

Typically coatings formulations will require a wetting agent or surfactant for the purpose of emulsification, compatibilization of components, leveling, flow and reduction of surface defects. Additionally, these additives may provide improvements in the cured or dry film, such as improved abrasion resistance, antiblocking, hydrophilic, and hydrophobic properties. Coatings formulations may exists as, Solvent-borne coatings, water-borne coatings and powder coatings.

The coatings components may be employed as: architecture coatings; OEM product coatings such as automotive coatings and coil coatings; Special Purpose coatings such as industrial maintenance coatings and marine coatings;

Typical resin types include: Polyesters, alkyds, acrylics, epoxies

### C. Personal Care

In a preferred embodiment, silicone compositions of the present invention comprises, per 100 parts by weight (“pbw”) of the personal care composition, from 0.1 to 99 pbw, more preferably from 0.5 pbw to 30 pbw and still more preferably from 1 to 15 pbw of the composition of the present invention and from 1 pbw to 99.9 pbw, more preferably from 70 pbw to 99.5 pbw, and still more preferably from 85 pbw to 99 pbw of the personal care composition.

The compositions of the present invention may be utilized in personal care emulsions, such as lotions, and creams. As is generally known, emulsions comprise at least two immiscible phases one of which is continuous and the other which is discontinuous. Further emulsions may be liquids with varying viscosities or solids. Additionally the particle size of the emulsions may render them microemulsions and, when sufficiently small, microemulsions may be transparent. Further it is also possible to prepare emulsions of emulsions and these are generally known as multiple emulsions.

These emulsions may be:

1) aqueous emulsions where the discontinuous phase comprises water and the continuous phase comprises silicone compositions of the present invention;

2) aqueous emulsions where the discontinuous phase comprises silicone compositions of the present invention and the continuous phase comprises water:

3) non-aqueous emulsions where the discontinuous phase comprises a non-aqueous hydroxylic solvent and the continuous phase comprises silicone compositions of the present invention; and

4) non-aqueous emulsions where the continuous phase comprises a non-aqueous hydroxylic organic solvent and the discontinuous phase comprises silicone compositions of the present invention.

Non-aqueous emulsions comprising a silicone phase are described in U.S. Pat. No. 6,060,546 and U.S. Pat. No. 6,271,295 the disclosures of which are herewith and hereby specifically incorporated by reference.

As used herein the term “non-aqueous hydroxylic organic compound” means hydroxyl containing organic compounds exemplified by alcohols, glycols, polyhydric alcohols and polymeric glycols and mixtures thereof that are liquid at room temperature, e.g. about 25° C., and about one atmosphere pressure. The non-aqueous organic hydroxylic solvents are selected from the group consisting of hydroxyl containing organic compounds comprising alcohols, glycols, polyhydric alcohols and polymeric glycols and mixtures thereof that are liquid at room temperature, e.g. about 25° C., and about one atmosphere pressure. Preferably the non-aqueous hydroxylic organic solvent is selected from the group consisting of ethylene glycol, ethanol, propyl alcohol, iso-propyl alcohol, propylene glycol, dipropylene glycol, tripropylene glycol, butylene glycol, iso-butylene glycol, methyl propane diol, glycerin, sorbitol, polyethylene glycol, polypropylene glycol mono alkyl ethers, polyoxyalkylene copolymers and mixtures thereof.

Once the desired form is attained whether as a silicone only phase, an anhydrous mixture comprising the silicone phase, a hydrous mixture comprising the silicone phase, a water-in-oil emulsion, an oil-in-water emulsion, or either of the two non-aqueous emulsions or variations thereon, the resulting material is usually a cream or lotion with improved deposition properties and good feel characteristics. It is capable of being blended into formulations for hair care, skin care, antiperspirants, sunscreens, cosmetics, color cosmetics, insect repellants, vitamin and hormone carriers, fragrance carriers and the like.

The personal care applications where silicone compositions of the present invention may be employed include, but are not limited to, deodorants, antiperspirants, antiperspirant/deodorants, shaving products, skin lotions, moisturizers, toners, bath products, cleansing products, hair care products such as shampoos, conditioners, mousses, styling gels, hair sprays, hair dyes, hair color products, hair bleaches, waving products, hair straighteners, manicure products such as nail polish, nail polish remover, nails creams and lotions, cuticle softeners, protective creams such as sunscreen, insect repellent and anti-aging products, color cosmetics such as lipsticks. foundations, face powders, eye liners, eye shadows, blushes, makeup, mascaras and other personal care formulations where silicone components have been conventionally added, as well as drug delivery systems for topical application of medicinal compositions that are to be applied to the skin.

In a preferred embodiment, the personal care composition of the present invention further comprises one or more personal care ingredients. Suitable personal care ingredients include, for example, emollients, moisturizers, humectants, pigments, including pearlescent pigments such as, for example, bismuth oxychloride and titanium dioxide coated mica, colorants, fragrances, biocides, preservatives, antioxidants, anti-microbial agents, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, organic oils, waxes, film formers, thickening agents such as, for example, fumed silica or hydrated silica, particulate fillers, such as for example, talc, kaolin, starch, modified starch, mica, nylon, clays, such as, for example, bentonite and organo-modified clays.

Suitable personal care compositions are made by combining, in a manner known in the art, such as, for example, by mixing, one or more of the above components with the compositions of the present invention. Suitable personal care compositions may be in the form of a single phase or in the form of an emulsion, including oil-in-water, water-in-oil and anhydrous emulsions where the silicone phase may be either the discontinuous phase or the continuous phase, as well as multiple emulsions, such as, for example, oil-in water-in-oil emulsions and water-in-oil-in water-emulsions.

In one useful embodiment, an antiperspirant composition comprises silicone compositions of the present invention and one or more active antiperspirant agents. Suitable antiperspirant agents include, for example, the Category I active antiperspirant ingredients listed in the U.S. Food and Drug Administration's Oct. 10, 1993 Monograph on antiperspirant drug products for over-the-counter human use, such as, for example, aluminum halides, aluminum hydroxyhalides, for example, aluminum chlorohydrate, and complexes or mixtures thereof with zirconyl oxyhalides and zirconyl hydroxyhalides, such as for example, aluminum-zirconium chlorohydrate, aluminum zirconium glycine complexes, such as, for example, aluminum zirconium tetrachlorohydrex gly.

In another useful embodiment, a skin care composition comprises the compositions of the present invention, and a vehicle, such as, for example, a silicone oil or an organic oil. The skin care composition may, optionally, further include emollients, such as, for example, triglyceride esters, wax esters, alkyl or alkenyl esters of fatty acids or polyhydric alcohol esters and one or more the known components conventionally used in skin care compositions, such as, for example, pigments, vitamins, such as, for example, Vitamin A, Vitamin C and Vitamin E, sunscreen or sunblock compounds, such as, for example, titanium dioxide, zinc oxide, oxybenzone, octylmethoxy cinnamate, butylmethoxy dibenzoylmethane, p-aminobenzoic acid and octyl dimethyl-p-aminobenzoic acid.

In another useful embodiment, a color cosmetic composition, such as, for example, a lipstick, a makeup or a mascara composition comprises the compositions of the present invention, and a coloring agent, such as a pigment, a water soluble dye or a liposoluble dye.

In another useful embodiment, the compositions of the present invention are utilized in conjunction with fragrant materials. These fragrant materials may be fragrant compounds, encapsulated fragrant compounds, or fragrance releasing compounds that either the neat compounds or are encapsulated. Particularly compatible with the compositions of the present invention are the fragrance releasing silicon containing compounds as disclosed in U.S. Pat. Nos. 6,046,156; 6,054,547, 6,075,111; 6,077,923; 6,083,901; and 6,153,578; all of which are herein and herewith specifically incorporated by reference.

The uses of the compositions of the present invention are not restricted to personal care compositions, other products such as waxes, polishes and textiles treated with the compositions of the present invention are also contemplated.

### D. Home Care

Home care applications include laundry detergent and fabric softener, dishwashing liquids, wood and furniture polish, floor polish, tub and tile cleaners, toilet bowl cleaners, hard surface cleaners, window cleaners, antifog agents, drain cleaners, auto-dish washing detergents and sheeting agents, carpet cleaners, prewash spotters, rust cleaners and scale removers.

### E. Oil and Gas

Compositions of the present invention are useful in oil and gas applications, including demulsification.

### F. Water Processing

Compositions comprising the present invention are useful for applications involving commercial and industrial open recirculating cooling water towers, closed cooling water systems, cooling water conduits, heat exchangers, condensers, once-through cooling systems, Pasteurizers, air washers, heat exchange systems, air conditioning/humidifiers/dehumidifiers, hydrostatic cookers, safety and/or fire water protection storage systems, water scrubbers, disposal wells, influent water systems, including filtration and clarifiers, wastewater treatment, wastewater treatment tanks, conduits, filtration beds, digesters, clarifiers, holding ponds, settling lagoons, canals, odor control, ion exchange resin beds, membrane filtration, reverse osmosis, micro- and ultra-filtration, assisting in the removal of biofilms in cooling tower applications, heat exchangers and process water systems, and the like.

### G. Pulp and Paper

Compositions of the present invention are useful in pulp and paper applications, such as paperboard defoamers, and wetting agents for the pulping process.

### H. Rubbers

Compositions of the present invention are useful for rubber applications such as tires, hoses, transmission belts, conveyor belts, conveyor belt covers, wiper blades, shoes, shoe soles, rubber-lined cloths, packing, lining, protective coatings, general purpose sheeting, electrical cable insulation, roofing materials, flooring materials, aerospace, computer, advanced mining operations, and automotive parts.

### I. Drug Delivery System

Transdermal drug delivery systems may be designed to act locally at the point of application or to act systemically by entering the body's blood circulation. In these systems, delivery may be achieved by direct topical application of a substance or drug in the form of an ointment or the like, or by adhesion of a patch with a reservoir that holds the drug and releases it to the skin in a time-controlled fashion.

The drug to be delivered, can include, without limitation, an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist or an antioxidant or any combination thereof.

Examples of antiproliferative agents include, without limitation, actinomycin D, or derivatives or analogs thereof, e.g., actinomycin I₁, actinomycin actinomycin X₁, and actinomycin C₁. Antiproliferative agents can be natural proteineous agents such as cytotoxins or they can be natural or synthetic small molecules such as, without limitation, taxoids such as taxols, docetaxel, paclitaxel and paclitaxel derivatives, macrolide compounds such as, without limitation, rapamycin and derivative and analogs thereof such as everolimus, biolimus and tacrolimus and derivatives of any of the foregoing perfenidone and prodrugs and co-drugs of any of the foregoing thereof as well as combinations of any of these. Additional rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin, prodrugs thereof, co-drugs thereof and combinations thereof.

Examples of anti-inflammatory agents include, without limitation, steroidal and nonsteroidal anti-inflammatory compounds such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamnic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin, salicylic acid, corticosteroids, glucocorticoids, tacrolimus, pimecrolimus, prodrugs, co-drugs and combinations thereof. The anti-inflammatory agent may also be a biological inhibitor of proinflammatory signaling molecules such as antibodies that bind to such signaling molecules.

Examples of antineoplastics and antimitotics include, without limitation, paclitaxel, docetaxel, methotrexate, azathioprine, vincristine, vinblastine, fluorouracil, doxorubicin hydrochloride and mitomycin.

Examples of antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, recombinant hirudin and thrombin, thrombin inhibitors, calcium channel blockers (such as nifedipine), colchicine, fish oil (omega 3-fatty acid), histamine antagonists, lovastatin, monoclonal antibodies, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as vitamins, and combinations thereof.

An example of an antiallergic agent is permirolast potassium.

Examples of potentially useful cytostatic or antiproliferative agents include, without limitation, angiopeptin, angiotensin converting enzyme inhibitors such as captopril, cilazapril or lisinopril, calcium channel blockers such as nifedipine; colchicine; fish oil (omega-3-fatty acid); histamine antagonists; lovastatin, monoclonal antibodies; nitroprusside, phosphodiesterase inhibitors, prostaglandin inhibitors, suramin, serotonin blockers, steroids and thioprotease inhibitors.

Some additional potentially useful drugs include, without limitation, any bioactive synthetic inorganic and organic compounds, proteins and peptides, polysaccharides and other sugars, lipids, and DNA and RNA nucleic acid sequences having therapeutic, prophylactic or diagnostic activities, nucleic acid sequences include genes, antisense molecules which bind to complementary DNA to inhibit transcription, ribozymes, antibodies, receptor ligands, enzymes, adhesion peptides, blood clotting factors, inhibitors or clot dissolving agents, antigens for immunization, oligonucleotides such as antisense oligonucleotides and ribozymes and retroviral vectors for use in gene therapy; antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents.

In one embodiment, a method of producing the silicone compositions described herein comprising contacting at least one hydride-functional silicone (i), at least one unsaturated-functional silicone (ii) and at least one epoxy or oxetane functional silicone (iii) to produce the silicone composition.

In one embodiment, a method of producing the silicone compositions described herein comprising (a) contacting at least one hydride-functional silicone (i) and at least one unsaturated-functional silicone (ii), and (b) adding at least one epoxy or oxetane functional silicone (iii) to produce the silicone composition.

In one embodiment, a method of producing the silicone compositions described herein comprising (a) contacting at least one hydride-functional silicone (i) and at least one epoxy or oxetane functional silicone (iii), and (b) adding at least one unsaturated-functional silicone (ii) to produce the silicone composition.

In one embodiment, a method of producing the silicone compositions described herein comprising (a) contacting at least one unsaturated-functional silicone (ii) and at least one epoxy or oxetane functional silicone (iii), and (b) adding at least one hydride-functional silicone (i) to produce the silicone composition.

In one embodiment, a method of producing the silicone compositions described herein comprising (a) contacting at least one hydride-functional silicone (i) and at least one unsaturated-functional silicone (ii) to produce a silicone (i)/(ii) stock; (b) contacting at least one hydride-functional silicone (i) and at least one epoxy or oxetane functional silicone (iii) to produce a silicone (i)/(iii) stock; (c) contacting the silicone (i)/(ii) stock and the silicone (i)/(iii) stock to produce a silicone composition.

In one embodiment, a method of producing the silicone compositions described herein comprising (a) contacting at least one hydride-functional silicone (i) and at least one unsaturated-functional silicone (ii) to produce a silicone (i)/(ii) stock; (b) contacting at least one unsaturated-functional silicone (ii) and at least one epoxy or oxetane functional silicone (iii) to produce a silicone (ii)/(iii) stock; (c) contacting the silicone (i)/(ii) stock and the silicone (ii)/(iii) stock to produce a silicone composition.

In one embodiment, a method of producing the silicone compositions described herein comprising (a) contacting at least one hydride-functional silicone (i) and at least one epoxy or oxetane functional silicone (iii) to produce a silicone (i)/(iii) stock; (b) contacting at least one unsaturated-functional silicone (ii) and at least one epoxy or oxetane functional silicone (iii) to produce a silicone (ii)/(iii) stock; (c) contacting the silicone (i)/(iii) stock and the silicone (ii)/(iii) stock to produce a silicone composition.

In one embodiment, hydride-functional polysiloxane such as silicone (i) is utilized to crosslink both unsaturated-functional polysiloxane such as silicone (ii) and epoxy or oxetane functional polysiloxane such as silicone (iii) into the reaction product concurrently. The unsaturated-functional polysiloxane (ii) undergoes a hydrosilylation reaction, i.e., the addition of Si—H bonds across unsaturated bonds, e.g., believed to occur according to a modified Chalk-Harrod cycle.

In such a Chalk-Harrod cycle, an oxidative addition of a vinyl silicone to a metal complex is followed by a migratory insertion of a hydride silicone, and the resulting complex undergoes reductive elimination and regeneration of the metal complex.

The epoxy or oxetane functional polysiloxane undergoes an epoxide ring-opening reaction, in which the cationic ring opening polymerization of epoxy (or oxetane) groups occurs. The epoxy or oxetane functional polysiloxane can also optionally crosslink to itself through a ring-opening reaction. These two curing reactions occurring concurrently provides the dual modalities of the curing system, the dual modality cure system, i.e., a cure of both the noted hydride-unsaturated reaction(s) and the noted hydride-epoxy (or oxetane) reaction(s), being activated, in one embodiment, by either ultraviolet light or heat or both, in the presence of a catalyst such as a platinum catalyst.

While not wishing to be bound by any particular theory, it is believed that when an unsaturated-functional polysiloxane such as silicone (i), hydride-functional polysiloxane such as silicone (ii) and epoxy or oxetane functional polysiloxane such as silicone (iii) are blended together and vulcanize in the presence of platinum catalyst, the hydrosilylation reaction and the epoxy (or oxetane) ring opening reaction both occur concurrently. A portion of the platinum catalyst is released from the Chalk-Harrod cycle by reacting with epoxide and this forms a new active site to grow an ether or polyether chain, while the rest of the platinum catalyst stays in the Chalk-Harrod cycle to continue the hydrosilylation reaction. A simple mechanism for this dual cure system is illustrated in the scheme below. The resulting reaction product, in one embodiment, has enhanced

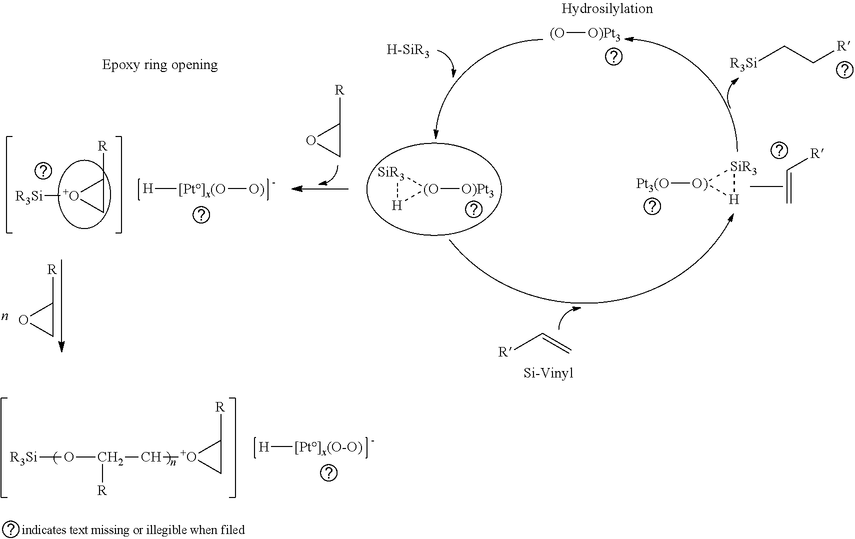

In one embodiment herein, the unsaturated-functional silicones are used in the amount of from about 0.1 weight (wt %) to about 99 wt % of the total weight of the composition, more specifically of from about 50 wt % to about 95 wt %, and most specifically of from about 60 wt % to about 85 wt %. The hydride-functional silicones are used in the amount of from about 1 wt % to about 50 wt % of the total weight of the composition, more specifically of from about 3 wt % to about 15 wt %, and most specifically of from about 5 wt % to about 10 wt %. The epoxy or oxetane functional silicones are used in the amount of from about 0.1 wt % to about 98 wt % of the total weight of the composition, more specifically of from about 3 wt % to about 40 wt %, and most specifically of from about 10 by wt % to about 30 wt %.

In another embodiment, the photo-initiator is used in the amount of from about 0 to about 2 wt % of the total weight of the composition. The silica filler is used in the amount of from 0 to about 50 wt % of the total weight of the composition, more specifically of from about 0 wt % to about 40 wt %, and most specifically of from about 0 to about 30 wt %.

In a further embodiment, the platinum catalyst can be employed in amount of from about 0.1 parts per million (ppm) to about 500 ppm of element platinum, more specifically of from about 1 ppm to about 60 ppm, and most specifically of from about 2 ppm to about 30 ppm.

In one embodiment, the mixture was cured under a UV lamp with >25% of the UV power in the range of 200-400 nm allocated within the UVA range. i.e., 320-400 nm, more specifically >65% of the UV power allocated within the UVA range, and most specifically >99% of the UV power allocated within the UVA range. In one specific embodiment, the mixture was cured at a UVA radiation dose of >0.5 J/cm², more specifically at a UVA radiation dose of >2 J/cm², and most specifically at a UVA radiation dose of >5 J/cm².

Depending on the relative amounts of D, D^{P} and D^{E} groups in the final cross-linked copolymeric network composition, the cross-linked composition will be swellable by either 1) a hydroxylic solvent such as water, an alcohol, or a carboxylic acid or solvent mixture where an aqueous or non-aqueous hydroxylic solvent is a component or 2) a non-aqueous non-hydrophilic solvent that may either be a silicone or an organic solvent as hereinafter later defined, or mixtures containing such solvents. For purposes of this discussion only these two classes of cross-linked swellable network copolymers will be referred to as “water-swellable” or “oil-swellable” (the term oil swellable encompassing all swelling solvents not embraced by the term “water swellable”). Generally, water swellability is more likely to occur with cross-linked network copolymers where the following relationships obtain: (1) for the number of D groups present: about 5<number of D groups<about 90; (2) for the number of D^{P} groups present: D^{P}>about 5; and (3) the acrylate cross-links constituting at least about 5 weight percent or more of the non-swollen cross-linked polymer network.

In contrast, oil swellability is more likely to occur with cross-linked network copolymers where the following relationships obtain: (1) for the number of D groups present: about 90≦number of D groups; (2) for the number of D^{P} groups present: about 1<number of D^{P} groups≦about 7; and (3) the acrylate cross-links constituting no more than about 10 weight percent or less of the non-swollen cross-linked polymer network.

It is to be emphasized that the preceding ranges of structural parameters and stoichiometric subscripts exemplified for water or oil swellability are variable and interdependent and each parametric variable may be exceeded by being greater than or less than the indicated ranges and still observing a particular type of swellability by reason of a homeostatic variation in another structural or stoichiometric parameter associated with the particular polymer.

Since the polyether substituents of the polymer or copolymer are capable of hydrogen bonding with water and other hydroxylic solvents, increasing the content of such polyether substituents, all other composition variables remaining constant, will tend to increase the water swellability of the resulting cross-linked network polymer. Because it is possible to vary the compositional parameters of the cross-linked network copolymers of the invention in an almost limitless fashion, some compositions will be both water swellable and oil swellable while others will only be water swellable or oil swellable, and some compositions will not be swellable with any of the solvents discussed herein. The amount of crosslinking present in the crosslinked network may be characterized with respect to the degree of swelling exhibited by the network in the fluid. In another embodiment, the crosslinked structure of the network is effective to allow the network to be swollen from its original volume to a swollen volume that is a factor of from 1.01 to 5000, more preferably from 2 to 1000, and even more preferably from 5 to 500, times its original volume. The original volume of the network can be determined, for example, by extracting or evaporating all of the internal fluid component from the silicone composition of the present invention to leave the original volume, that is, the volume of the copolymer network in the absence of the fluid.

### EXAMPLES

Intermediate Samples 1-4 were prepared as intermediate binary blends of an epoxy-functional polysiloxane having the formula MD^{E}₁₀D₄₉₀M with various hydride-functional polysiloxanes shown in Table 1. Trimethyl(methylcyclopentadienyl) platinum(IV) (CpPt) was mixed with an epoxy-functional polysiloxane having the formula M^{E}D^{E}₄D₉₅M^{E}. CpPt was utilized to photo-catalyze the epoxide ring-opening reaction between epoxy silicone and hydride silicone upon exposure to UV irradiation. The hydride/epoxy molar ratio was maintained at 2:1, while platinum level was kept at 6 ppm.

Intermediate Samples 1-4 were thoroughly mixed using a benchtop SpeedMixer. Approximately 11-12 gram of each sample were then poured into aluminum weighing dishes and cured in Fusion UV Curing Conveyor System (Model DRR-120). The UVA (320-400 nm) dosage received by the samples was measured using UV Power Puck II. As can be seen from Table 1, significantly less UVA dosage was needed for curing the samples with higher hydride functionality, e.g., Samples 1 and 2 vs Sample 3. When there were only two hydride functional groups per polysiloxane chain (i.e., Sample 4), the sample failed to cure.

TABLE 1
Intermediate binary blends of epoxy and hydride silicones

| | | | | |
|---|---|---|---|---|
| Sample No. | 1 | 2 | 3 | 4 |

Formulation

| | | | | |
|---|---|---|---|---|
| MD^{E}₁₀D₄₉₀M (0.26 mmol/g epoxy) (g) | 95.5 | 92.3 | 80.9 | 71.5 |
| CpPt in M^{E}D^{E}₄D₉₅M^{E} (600 ppm Pt) (g) | 1 | 1 | 1 | 1 |
| MD^{H}₂₀M (14.7 mmol/g H) (g) | 3.5 | | | |
| MD₂₀D^{H}₂₀M (7.4 mmol/g H) (g) | | 6.7 | | |
| MD₁₇D^{H}₄M (2.4 mmol/g H) (g) | | | 18.1 | |
| M^{H}D₁₇M^{H} (1.4 mmol/g H) (g) | | | | 27.5 |

Curing Property

| | | | | |
|---|---|---|---|---|
| UVA dosage to cure (J/cm²) | 7 | 7 | >16 | Not cured |

Wherein M is (CH₃)₃SiO_{1/2}, M^{E} is (CH₃)₂R^{E}SiO_{1/2}, M^{H} is (CH₃)₂HSiO_{1/2}, D is (CH₃)₂SiO_{2/2}, D^{E} is CH₃R^{E}SiO_{2/2}, D^{H} is CH₃HSiO_{2/2}, and R^{E} is 2-(3,4-epoxycyclohexyl)ethyl.

Examples 5-10 were prepared as ternary blends of vinyl, epoxy and hydride silicones. The vinyl silicone/hydride silicone (V/H) stock was prepared by blending M^{Vi}D₅₄₀M^{Vi}, M^{Vi}D₉₀₀M^{Vi} and MD₂₀D^{H}₂₀M at a ratio of 54:44:2 by weight, wherein M^{Vi} is (CH₃)₂(CH═CH₂)SiO_{1/2}, D is (CH₃)₂SiO_{2/2} and D^{H} is CH₃HSiO_{2/2}. The epoxy silicone/hydride silicone (E/H) stock was prepared by blending M^{E}D^{E}₄D₉₅M^{E} and MD₂₀D^{H}₂₀M at a ratio of 75:25 by weight wherein M^{E} is (CH₃)₂R^{E}SiO_{1/2}, D^{E} is CH₃R^{E}SiO_{2/2}, D is (CH₃)₂SiO_{2/2} and R^{E} is 2-(3,4-epoxycyclohexyl)ethyl. The platinum/vinyl silicone (Pt/SiVi) stock I was prepared by dissolving 25 milligram (mg) of CpPt in 50 gram (g) of M^{Vi}D₅₄₀M^{Vi}, while platinum/epoxy silicone (Pt/SiE) stock was prepared by dissolving 49 mg of CpPt in 50 g of M^{E}D^{E}₄D₉₅M^{E}.

Examples 5-10 were mixed thoroughly using a benchtop SpeedMixer. The samples were poured into a 3 millimeter (mm) thick 6 inch by 6 inch steel frame placed on top of a Plexiglass acrylic sheet, then smoothed using a bare steel rod, and cured in the Fusion UV Curing Conveyor System. The fully cured sample slabs were subject to different physical, mechanical and optical measurements, the results are shown in Table 2.

TABLE 2
Ternary blends of vinyl, epoxy and hydride silicones

| Example No. | | | | | |
|---|---|---|---|---|---|
| 5 | 6 | 7 | 8 | 9 | 10 |

Formulation

| | | | | | | |
|---|---|---|---|---|---|---|
| SiVi/SiH stock | 90 | 85.5 | 67.5 | 45 | 22.5 | |
| SiE/SiH stock | | 4.5 | 22.5 | 42.3 | 64.8 | 87.3 |
| Pt/SiVi stock I | 1.8 | 1.8 | 1.8 | | | |
| Pt/SiE stock | | | | 2.7 | 2.7 | 2.7 |

Properties

| SiE/SiH (wt %) | | 0 | 5% | 25% | 50% | 75% | 100% |
|---|---|---|---|---|---|---|---|
| Shore A Hardness (ASTM D2240) | | 20 | 20 | 25 | 43 | F* | F* |
| Tensile Strength (ASTM D638) (psi) | | 60 | 60 | 250 | 100 | 40 | F* |
| Elongation at Break (ASTM D638) | | 130 | 150 | 200 | 50 | 10 | F* |
| (%) | | | | | | | |
| Water Uptake at 500 h (wt %) | | 0.03% | 0.07% | 0.29% | 0.37% | 0.53% | 0.70% |
| Haze at 3 mm (%) | Initial | 2.1 | 2.1 | 2.2 | 3.9 | 6.7 | 0.8 |
| | 500 h in Water | 32 | 33 | 27 | 8.8 | 9.6 | 2.4 |
| | Change | 30 | 31 | 25 | 4.9 | 2.9 | 1.6 |
| Transmission at | Initial | 95.1 | 95.3 | 94.5 | 94.2 | 94.2 | 94.1 |
| 3 mm (%) | 500 h in Water | 93.1 | 93.8 | 93.5 | 93.7 | 93.8 | 93.9 |
| | Change | −2.0 | −1.5 | −1.0 | −0.5 | −0.4 | −0.2 |

*F: Fail to yield a reliable number as the sample gets fractured during the measurement.

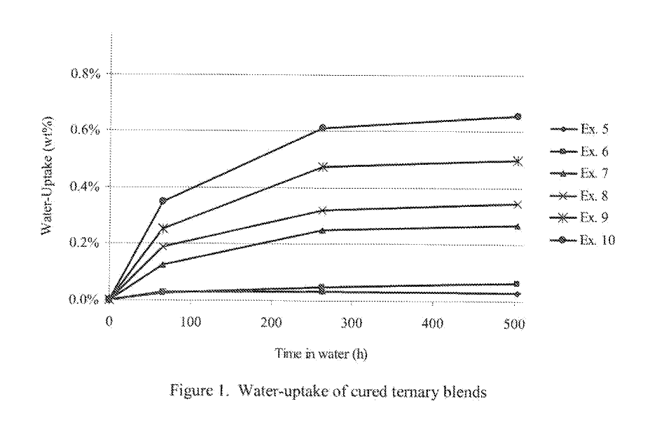

As illustrated in Table 2, the tensile strength and elongation are retained or improved when the epoxy silicone content is below 30 wt %, while the water uptake is increased significantly (see also FIG. 1), indicating enhanced hydrophilicity with the introduction of hydrophilic ether moiety in the composition. The water uptake continues to increase as the content of epoxy silicone increases; however, the cured composition samples also tend to get more brittle, and the tensile strength starts to drop.

In addition to improved hydrophilicity, the optical properties of the cured silicone composition are also enhanced with the introduction of an ether moiety. As shown in Table 2, when the samples were soaked in deionized water, the change of haze and transmission continued to drop as the epoxy silicone content increased. The % haze of a typical unfilled addition-cure silicone composition is about 2 at 3 mm. Example 5 has a % haze of 32, a dramatic increase from 2.1, when immersed in water for 500 hours, at which point the sample appeared cloudy. On the other hand, for a composition containing 76 wt % of epoxy polysiloxane, such as Example 10, the % haze only increased slightly from 0.8 to 2.4. The samples stayed clear throughout the water immersion.

Intermediate Samples 11-18 were prepared as binary blends of either epoxy-functional polysiloxane or vinyl-functional polysiloxane with hydride-functional polysiloxane shown in Table 3.

TABLE 3
Intermediate binary blends of epoxy/hydride silicones or vinyl/hydride silicones

| | Sample No. | | | | | | | |
|---|---|---|---|---|---|---|---|---|
| | #11 | #12 | #13 | #14 | #15 | #16 | #17 | #18 |
| Formulation | | | | | | | | |
| 60% M^{E}₃M₁₂T₁₀Q₁₀ + 40% | 72.4 | 76.1 | | | | | | |
| M^{E}D₂₅M^{E} (0.67 mmol/g epoxy) | | | | | | | | |
| (g) | | | | | | | | |
| M^{E}D^{E}_{3.8}D₉₄M^{E} (0.69 mmol/g | | | 71.9 | 75.7 | | | | |
| epoxy) (g) | | | | | | | | |
| 60 wt % M^{Vi}_{0.15}M_{0.87}Q + 40 wt % | | | | | 76.2 | 79.5 | | |
| M^{Vi}D₁₄₀M^{Vi} (0.56 mmol/g Vi) (g) | | | | | | | | |
| 55% M^{Vi}D₅₄₀M^{Vi} + 45% | | | | | | | 95.08 | 95.44 |
| M^{Vi}D₉₀₀M^{Vi} (0.04 mmol/g Vi) (g) | | | | | | | | |
| MD₂₀D^{H}₂₀M (7.4 mmol/g H) (g) | 24.6 | | 25.1 | | 20.8 | | 1.92 | |
| M^{H}_{1.7x}Qₓ (9.15 mmol/g H) (g) | | 20.9 | | 21.3 | | 17.5 | | 1.56 |
| Pt/SiE stock (g) | 3.0 | 3.0 | 3.0 | 3.0 | | | | |
| Pt/SiV stock II (g) | | | | | 3.0 | 3.0 | 3.00 | 3.00 |
| Curing Property | | | | | | | | |
| UVA radiation dose needed to | 18 | Not | 7 | >112* | 1.6 | 2.2 | 0.6 | 1.2 |
| cure under metal halide UV lamp | | cured | | | | | | |
| (J/cm²) | | | | | | | | |

*Gel formation starts at 32 J/cm² of UVA radiation dose; however, full vulcanization is still not yet reached at 112 J/cm² of UVA radiation dose; and wherein M is (CH₃)₃SiO_{1/2}, M^{E} is (CH₃)₂R^{E}SiO_{1/2}, M^{Vi} is (CH₃)₂(CH═CH₂)SiO_{1/2}, M^{H} is (CH₃)₂HSiO_{1/2}, D is (CH₃)₂SiO_{2/2}, D^{E} is CH₃R^{E}SiO_{2/2}, D^{H} is CH₃HSiO_{2/2} , T is CH₃SiO_{3/2}, Q is SiO_{4/2} and R^{E} is 2-(3,4-epoxycyclohexyl)ethyl.

Pt/SiV stock II was prepared by dissolving 49 mg of CpPt in 50 g of M^{Vi}D₅₄₀M^{Vi}. CpPt was utilized to photo-catalyze both epoxide ring-opening reaction between epoxy and hydride silicones and hydrosilylation reaction between vinyl and hydride silicones upon UV radiation. The hydride/epoxy molar ratio or hydride/vinyl molar ratio was held at 3.6:1, while platinum level was maintained at 18 ppm.

Intermediate Samples 11-18 were mixed thoroughly on a benchtop SpeedMixer. Approximately 12 g of each sample were then poured into aluminum weighing dishes and cured in Dymax 5000 Flood UV Curing Unit where a metal halide lamp was utilized as the UV source. The UVA (320-400 nm) radiation dose received by the samples was measured using UV Power Puck II.

As can be seen from Table 3, silicone polymers without the presence of T or Q units, regardless of whether epoxy, vinyl or hydride silicones were used, had fast kinetics in both epoxide ring opening and hydrosilylation reactions. As a result, a relatively higher UVA radiation dose was needed to cure silicone polymers having T or Q units, as shown in Sample 1 vs Sample 13, Sample 14 vs Sample 13 for epoxide ring opening, Sample 15 vs Sample 17, Sample 18 vs Sample 17, Sample 16 vs Sample 18, and Sample 16 vs Sample 15 for hydrosilylation.

Examples 19-24 were prepared as ternary blends of epoxy, vinyl and hydride silicones, as shown in Table 4. They were prepared as mixtures of two different binary blends at a ratio of 50:50 by weight.

TABLE 4
Ternary blends of epoxy, vinyl and hydride silicones

| | Example No. | | | | | |
|---|---|---|---|---|---|---|
| | #19 | #20 | #21 | #22 | #23 | #24 |
| Formulation | | | | | | |
| Sample #11 (g) | | 50 | 50 | | | |
| Sample #12 (g) | 50 | | | | | |
| Sample #13 (g) | | | | 50 | | 50 |
| Sample #14 (g) | | | | | 50 | |
| Sample #15 (g) | | 50 | | 50 | | |
| Sample #16 (g) | 50 | | | | | |
| Sample #17 (g) | | | 50 | | | 50 |
| Sample #18 (g) | | | | | 50 | |
| Curing Property | | | | | | |
| UVA radiation dose | 5.5* | 3 | 3 | 4 | 64* | 4 |
| needed to cure under | | | | | | |
| metal halide UV lamp | | | | | | |
| (J/cm²) | | | | | | |

*Tacky gel formed upon curing

Examples 19-24 were mixed thoroughly on a benchtop SpeedMixer. The samples were poured into aluminum weighing dishes and then cured in Dymax 5000 Flood UV Curing Unit. Examples 19-24 were vulcanized upon UV radiation. The UVA radiation dose needed to cure were found to be in between those required to vulcanize each individual binary blend.

Tacky gels, rather than cured elastomer slabs, was formed in Examples 19 and 23, indicating the formation of incomplete network. This result is expected, given that both Examples 19 and 23 contain a binary blend (Samples 12 and 14, respectively) that fails to reach full vulcanization upon UV radiation as high as 112 J/cm².

In one specific embodiment herein, the subject silicone compositions comprising at least one of the above-identified hydride-functional silicone (i), at least one of the above-identified unsaturated-functional silicone (ii) and at least one of the above-identified epoxy or oxetane functional silicone (iii) which silicones (i), (ii) and (iii) reacted optionally in the presence of a catalyst to produce the silicone composition and which silicone composition possesses dramatically improved hydrophilicity, physical properties and optical properties for uses in transdermal patches for healthcare and pharmaceutical applications, drug delivery devices, coating, cosmetic structuring material, gasketing materials and other applications where hydrophilicity is required.

These examples are to be construed as exemplary in nature only and are not intended in any way to limit the appended claims. It is contemplated that a person having ordinary skill in the art would be able to produce obvious variations of the subject matter and disclosures herein contained that would be by reason of such ordinary skill within the literal or equitable scope of the appended claims.

1. A silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1.

2. The silicone composition of claim 1, where R¹, R², R³, R⁴, R⁵, R⁶, R⁷ R⁸ and R⁹ are each independently selected from the group consisting of methyl, ethyl, isopropyl, sec-butyl and tert-butyl.

3. The silicone composition of claim 1, where each R^{vi} is independently selected from the group consisting of ethenyl, 2-propenyl, 3-butenyl, 5-hexenyl, 7-octenyl and ethenylphenyl.

4. The silicone composition of claim 1, where each R^{E} is independently selected from the group consisting of 2-(3,4-epoxycyclohexyl)ethyl, 2-(3,4-epoxycyclopentyl)ethyl, 2-(3,4-epoxycyclohexyl)propyl and 2-(3,4-epoxycyclopentyl)propyl.

5. The silicone composition of claim 1, where each R^{P} is an alkyl-poly(ethylene oxide-co-propylene oxide).

6. The silicone composition of claim 1, where the hydride-functional silicone (i) is used in the amount of from about 1 weight % to about 50 weight % of the total weight of the silicone composition.

7. The silicone composition of claim 1, where the hydride-functional silicone (i) has a hydride content in the range of from about 0.3 micromoles per gram to about 15 micromoles per gram.

8. The silicone composition of claim 1, where the unsaturated-functional silicone (ii) is used in the amount of from about 0.1 weight % to about 99 weight % of the total weight of the composition.

9. The silicone composition of claim 1, where the unsaturated-functional silicone (ii) has a unsaturated moiety content in the range of from about 0.01 micromoles per gram to about 2 micromoles per gram.

10. The silicone composition of claim 1, where the epoxy or oxetane functional silicone (iii) is used in the amount of from about 0.1 weight % to about 98 weight % of the total weight of the composition.

11. The silicone composition of claim 1, where the epoxy or oxetane functional silicone (iii) has an epoxy or an oxetane content in the range of from about 0.1 micromoles per gram to about 1.5 micromoles per gram.

12. The silicone composition of claim 1, further comprising a catalyst.

13. The silicone composition of claim 12, where the catalyst is a photo-activated platinum catalyst selected from the group consisting of η⁵-cyclopentadienyl platinum(IV) complexes, bis(β-diketonate) platinum(II) complexes, bis(phosphine) platinum(II) complexes, cyclooctadiene platinum(II) complexes, and mixtures thereof, preferably trimethyl(methylcyclopentadienyl) platinum(IV) (CpPt) or platinum(II) acetylacetonate (Pt(acac)2).

14. The silicone composition of claim 12, where the catalyst is a heat-activated platinum catalyst selected from the group consisting of platinum compounds such as chloroplatinic acid, or platinum complexes such as platinum/vinylsiloxane complexes, or mixtures thereof.

15. The silicone composition of claim 12, where the catalyst is a platinum catalyst having the content of element platinum in the amount of from about 0.1 parts per million to about 500 parts per million.

16. The silicone composition of claim 1, further comprising at least one of a photo-initiator, a filler, a photo-sensitizer, a stabilizer, an inhibitor and an adhesion promoter.

17. The silicone composition of claim 16, where the photo-initiator is selected from the group consisting of iron (II) sandwich complex of benzenecyclopentadienyliron (II) hexafluorophosphate, an iron (II) complex of cyclopentadienyl iron (II) dicarbonyl dimer, bis(4-alkylphenyl)iodonium salts of PF₆⁻, bis(4-alkylphenyl)iodonium salts of SbF₆⁻, and mixtures thereof.

18. The silicone composition of claim 16, where the filler is selected from the group consisting of fumed silica, precipitated silica and mixtures thereof.

19. The silicone composition of claim 1, wherein the reaction product is a cross-linked silicone polymer network.

20. The silicone composition of claim 1, wherein the reaction product is swellable and is hydrophilic.

21. A silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1;
wherein the reaction product has an enhanced hydrophilicity as compared to a reaction product that does not have the required respective functionalities.

22. A drug delivery device comprising:
1) a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1; and
2) at lease one active pharmaceutical ingredient.

23. A transdermal patch composition comprising:
at least one silicone layer comprising a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1.

24. A coating composition comprising:
a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1.

25. A cosmetic composition comprising:
1) a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1; and
2) a cosmetic ingredient.

26. The cosmetic composition according to claim 25 wherein said silicone composition may be swollen by a solvent.

27. The cosmetic composition according to claim 25 wherein said solvent is water.

28. The cosmetic composition according to claim 25 wherein said solvent is a silicone or an oil.

29. A gasketing composition comprising:
1) a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1; and
2) a filler.

30. A rubber composition comprising:
1) a silicone composition comprising the reaction product of:
a) at least one hydride-functional silicone having the formula (i)
MₐM^{H}_{b}M^{vi}_{c}M^{E}_{d}M^{P}ₑD_{f}D^{H}_{g}D^{vi}ₕD^{E}ᵢD^{P}ⱼTₖT^{H}ₘT^{vi}ₙT^{E}ₚT^{P}_{q}Qᵣ (i)
b) at least one unsaturated-functional silicone having the formula (ii) and
M_{a′}M^{H}_{b′}M^{vi}_{c′}M^{E}_{d′}M^{P}_{e′}D_{f′}D^{H}_{g′}D^{vi}_{h′}D^{E}_{i′}D^{P}_{j′}T_{k′}T^{H}_{m′}T^{vi}_{n′}T^{E}_{p′}T^{P}_{q′}Q_{r′} (ii)
c) at least one epoxy or oxetane functional silicone having the formula (iii)
M_{a″}M^{H}_{b″}M^{vi}_{c″}M^{E}_{d″}M^{P}_{e″}D_{f″}D^{H}_{g″}D^{vi}_{h″}D^{E}_{i″}D^{P}_{j″}T_{k″}T^{H}_{m″}T^{vi}_{n″}T^{E}_{p″}T^{P}_{q″}Q_{r″} (iii)
wherein:
M=R¹R²R³SiO_{1/2};
M^{H}=R⁷R⁸HSiO_{1/2};
M^{vi}=R¹⁰R¹¹R^{vi}SiO_{1/2};
M^{E}=R¹³R¹⁴R^{E}SiO_{1/2};
M^{P}=R¹⁶R¹⁷R^{P}SiO_{1/2};
D=R⁴R⁵SiO_{2/2};
D^{H}=R⁹HSiO_{2/2};
D^{vi}=R¹²R^{vi}SiO_{2/2};
D^{E}=R¹⁵R^{E}SiO_{2/2};
D^{P}=R¹⁸R^{P}Si_{2/2};
T=R⁶SiO_{3/2};
T^{H}=HSiO_{3/2};
T^{vi}=R^{vi}SiO_{3/2};
T^{E}=R^{E}SiO_{3/2};
T^{P}=R^{P}SiO_{3/2}; and
Q=SiO_{4/2};
where:
R¹, R², R³, R⁴ R⁵ and R⁶ are each independently monovalent hydrocarbon radicals having from 1 to about 60 carbon atoms;
R⁷, R⁸ and R⁹ are each independently hydrogen or monovalent hydrocarbon radicals having from 1 to about 60 carbon atoms;
R¹⁰, R¹¹ and R¹² are each independently monovalent hydrocarbon radicals having from 1 to about 60 carbon atoms or R^{vi};
each R^{vi} is independently a monovalent unsaturated hydrocarbon radical having from 2 to about 10 carbon atoms;
R¹³, R¹⁴ and R¹⁵ are each independently monovalent hydrocarbon radicals having from 1 to about 60 carbon atoms or R^{E};
each R^{E} is independently an epoxy or an oxetane radical having from 2 to about 60 carbon atoms, or having one or more heteroatoms;
R¹⁶, R¹⁷ and R¹⁸ are each independently a monovalent hydrocarbon radicals having from 1 to about 60 carbon atoms or R^{P};
each R^{P} is independently a monovalent polyether moiety (C_{S}H_{2S}O)x or a copolymer of monovalent polyether moieties (C_{S}H_{2S}O)_{X} and (C_{T}H_{2T}O)_{Y} or a higher order polymer wherein the subscripts S and T are either from 2 to about 20, and the subscripts X and Y are either from 1 to about 30;
the subscripts a, b, c, d, e, f, g, h, i, j, k, m, n, p, q and r are each either zero or positive integers from 1 to about 1000, subject to the limitations that a+b+c+d+e≧2, and b+g+m≧2;
the subscripts a′, b′, c′, d′, e′, f′, g′, h′, i′, j′, k′, m′, n′, p′, q′ and r′ are each either zero or positive integers from 1 to about 1000, subject to the limitations that a′+b′+c′+d′+e′≧2, and c′+h′+n′≧2; and
the subscripts a″, b″, c″, d″, e″, f″, g″, h″, i″, j″, k″, m″, n″, p″, q″ and r″ are each either zero or positive integers from 1 to about 1000, subject to the limitations that a″+b″+c″+d″+e″≧2, and d″+i″+p″≧1; and
2) a filler.
